SEQUENCE LISTING (1 sequence)

SEQ ID NO: 1 - protein, 8 aa, Artificial sequence
  Synthetic peptide
VAITVLVK

(19) United States
(12) Patent Application Publication
(10) Pub. No.: US 2021/0353564 A1
(43) Pub. Date: Nov. 18, 2021

(54) TREATMENTS FOR ZIKA VIRUS INFECTION

(71) Applicant: Saint Louis University, St. Louis, MO (US)

(72) Inventors: Christopher ARNATT, Manchester, MO (US); James BRIEN, St. Louis, MO (US); Amelia PINTO, St. Louis, MO (US)

(73) Assignee: Saint Louis University, St. Louis, MO (US)

(21) Appl. No.: 17/290,400

(22) PCT Filed: Oct. 28, 2019
(86) PCT No.: PCT/US2019/058263

§ 371 Date: Apr. 30, 2021

Related U.S. Application Data
(60) Provisional application No. 62/754,711, filed on Nov. 2, 2018.

(51) Int. Cl. A61K 31/138; A61K 45/06; A61P 31/14

(52) U.S. Cl. CPC A61K 31/138; A61P 31/14; A61K 45/06

(57) ABSTRACT

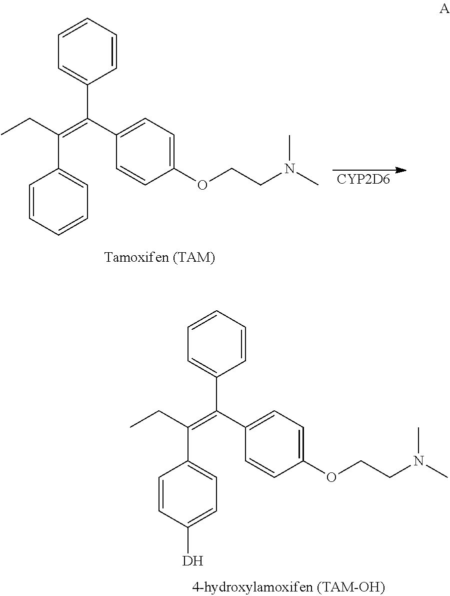

The present disclosure is directed to methods of treating Zika virus infections using selective estrogen receptor modulators (SERMs), such as Tamoxifen, or a compound selected from CALP-1, A7, PMA, Chelerythrine, PHTPP, Tariquidar or S-Equol.

### PRIORITY CLAIM

This application claims benefit of priority to U.S. Provisional Application Ser. No. 62/754,711, filed Nov. 2, 2018, the entire contents being hereby incorporated by reference.

### BACKGROUND

### 1. Field of the Disclosure

The present disclosure relates generally to the fields of medicine, infectious disease, and virology. More particular, the disclosure relates to the use of various chemical entities to treat Zika virus infection.

### 2. Background

Zika virus (ZIKV) is an emerging mosquito-transmitted flavivirus that has become a global public health threat. Recent ZIKV epidemics in Micronesia, Brazil, other parts of South and Central America, and Mexico (Duffy et al., 2009) are linked to Guillain-Barre syndrome in adults and microcephaly in newborn infants (Oehler et al., 2014; Musso et al., 2014) in the setting of infection during pregnancy (Araugo et al., 2016; Gatherer & Kohl, 2016). As ZIKV is transmitted by Aedes species mosquitoes, which are global in distribution, countries in which these vectors are present could be sites for future epidemics. Despite the potential for causing disease in millions, specific treatments or vaccines for ZIKV are not available, leaving a considerable unmet need in the field.

The selective estrogen receptor modulator (SERM) tamoxifen (TAM) has been shown to have antiviral activity against a variety of viruses including: HCV, human immunodeficiency virus (HIV), ebola virus, and herpes simplex virus. Watashi et al. (2007) have shown that TAM can suppress HCV replication through inhibiting the putative interaction between NSSB and estrogen receptor a (ERa). TAM and another SERM, ICI182780, were able to inhibit HCV genome replication without any apparent toxicity. This study thoroughly examined the interaction with ERa and NSSB and determined that the DNA-binding domain of ERa promotes the participation of NSSB in the NCV replication complex. Since the little is known how HCV proteins are regulated in the replication complex, this study could not elucidate the exact role of the ERa-NSSB interaction on HCV replication. Repurposing of other HCV antiviral strategies for ZIKV has yielded several new inhibitors of ZIKV replication, indicating the importance of looking at HCV targets in the hope of finding new ZIKV antivirals.

### SUMMARY

Thus, in accordance with the present disclosure, there is provided a method of treating a subject infected with Zika virus (ZIKV) or reducing the likelihood of infection of a subject at risk of contracting ZIKV, comprising delivering to said subject a selective estrogen receptor modulator (SERM) or a compound selected from CALP-1, A7, PMA, Chelerythrine, PHTPP, Tariquidar or S-Equol. The SERM may be MPP, Tamoxifen or 4-hydroxy Tamoxifen. The subject may be a pregnant female, a sexually active female, or a female undergoing fertility treatments, such as a human female.

Delivering may comprise administering 4-hydroxy Tamoxifen, or may comprise administering Tamoxifen, which is converted to 4-hydroxy Tamoxifen in said subject. Delivering may comprise oral, intra-venous or intra-arterial administration. The method may further comprise administering to said subject a second ZIKV therapy, such as a second SERM or palliative care. The SERM or a compound selected from CALP-1, A7, PMA, Chelerythrine, PHTPP, Tariquidar or S-Equol may be administered prior to infection or after infection

In another embodiment, there is provided a method of protecting the health of a placenta and/or fetus of a pregnant female subject, such as a human female, infected with or at risk of infection with Zika virus (ZIKV) comprising delivering to said subject a selective estrogen receptor modulator (SERM) or a compound selected from CALP-1, A7, PMA, Chelerythrine, PHTPP, Tariquidar or S-Equol. The SERM may be MPP, Tamoxifen or 4-hydroxy Tamoxifen. Delivering may comprise administering 4-hydroxy Tamoxifen, or may comprise administering Tamoxifen, which is converted to 4-hydroxy Tamoxifen in said subject.

Delivering may comprise oral, intra-venous or intra-arterial administration. The method may further comprise administering to said subject a second ZIKV therapy, such as a second SERM or palliative care. The SERM or a compound selected from CALP-1, A7, PMA, Chelerythrine, PHTPP, Tariquidar or S-Equol may be administered prior to infection or after infection. The SERM or a compound selected from CALP-1, A7, PMA, Chelerythrine, PHTPP, Tariquidar or S-Equol may increase the size of the placenta as compared to an untreated control and/or the SERM or a compound selected from CALP-1, A7, PMA, Chelerythrine, PHTPP, Tariquidar or S-Equol may reduce viral load and/or pathology of the fetus as compared to an untreated control.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number. It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Structure and Function of TAM-OH. (FIG. 1A) The structure and converstion of TAM to TAM-OH. (FIG. 1B) The effective concentration of TAM-OH on ZIKV strain PRVABC59 on Vero cells as determined by a focus reduction test. Average+/−StdDev from three independent experiments done in duplicate.

FIGS. 2A-B. Efficacy of TAM-OH in a mouse model of ZIKV disease. For both experiments, mice were treated on days −1, 1, and 2 with TAM-OH at 1 mg/mouse/day. (FIG. 2A) Weight change after sub-lethal infection of IFNAR mice with ZIKV. Five mice per group. (FIG. 2B) Survival of Ifnar−/−mice fol-lowing inoculation with 105 FFU of ZIKV by in-travenous injection. (n=4 vehicle, n=5 TAM-OH). Survival differences were statistically sig-nificant (*, p=0.02) as determined by Mantel-Cox test.

FIGS. 3-14. Focus Forming Assay. Data represent Vero cells drugged with SERM compounds an hour before addition of virus (T-1), upon addition of virus (T0), one hour after addition of virus (T1), eight hours after addition of virus (T8), and twelve hours after addition of virus. Each compound was screened as triplicate measurements and performed in duplicate. The data is shown as focus count (normalized to the control wells) which represents the amount of remaining virus after 39 hours as a function of log concentration. The twelve-hour time point has been replicated (data not shown).

FIGS. 15-24. Cytotoxicity Analysis. Cytotoxicity data collected using a crystal violet assay upon completion of the Focus Forming Assay. These data have since been verified using an MTS assay independent of the Focus Forming Assay (data not shown). To each well was added crystal violet and allowed to stain and the absorbance was measured at 590 nm after washing and compound wells were compared to control wells to get the percent viability.

FIG. 25. SI values calculated for EC₅₀+CC₅₀. The higher the value shown the less effect due to toxicity.

### DESCRIPTION OF ILLUSTRATIVE EMBODIMENTS

As discussed above, Zika virus (ZIKV) infection causes systemic and central nervous system pathology or disease, with congenital birth defects linked to infection during pregnancy (Coyne et al., 2016). Currently there are no approved vaccines or specific therapies to treat or prevent Zika virus (ZIKV) infection, and with its link to severe diseases such as congenital Zika syndrome and Guillain-Barre syndrome, there is an essential need for ZIKV therapeutics. Drug repurposing of current therapies for ZIKV is a possible way to address the urgent need for ZIKV antivirals, and using this approach, the inventors determined that the active metabolite of Tamoxifen, 4-hydroxy Tamoxifen (TAM-OH), is capable of reducing ZIKV infection in cultured cells at clinically relevant concentrations and leads to a reduction in weight loss in a mouse model of ZIKV disease. Thus, selective estrogen receptor modulators (SERMs) like TAM and TAM-OH are proposed as a new treatment for ZIKV infection. In addition, the inventors have expanded their search and identified a series of distinct compounds outside the SERM family that also inhibit ZIKV replication and reduce cytotoxicity caused by the virus.

This and other aspects of the disclosure are described in detail below.

### I. Zika Virus

Zika virus (ZIKV) is a member of the virus family Flaviviridae. It is spread by daytime-active Aedes mosquitoes, such as A. aegypti and A. albopictus. Its name comes from the Zika Forest of Uganda, where the virus was first isolated in 1947. Zika virus is related to the dengue, yellow fever, Japanese encephalitis, and West Nile viruses. Since the 1950s, it has been known to occur within a narrow equatorial belt from Africa to Asia. From 2007 to 2016, the virus spread eastward, across the Pacific Ocean to the Americas, leading to the 2015-16 Zika virus epidemic.

The infection, known as Zika fever or Zika virus disease, often causes no or only mild symptoms, similar to a very mild form of dengue fever. While there is no specific treatment, paracetamol (acetaminophen) and rest may help with the symptoms. As of 2016, the illness cannot be prevented by medications or vaccines. Zika can also spread from a pregnant woman to her fetus. This can result in microcephaly, severe brain malformations, and other birth defects. Zika infections in adults may result rarely in Guillain-Barré syndrome.

In January 2016, the United States Centers for Disease Control and Prevention (CDC) issued travel guidance on affected countries, including the use of enhanced precautions, and guidelines for pregnant women including considering postponing travel. Other governments or health agencies also issued similar travel warnings, while Colombia, the Dominican Republic, Puerto Rico, Ecuador, El Salvador, and Jamaica advised women to postpone getting pregnant until more is known about the risks.

The Zika virus belongs to the Flaviviridae family and the Flavivirus genus, and is thus related to the dengue, yellow fever, Japanese encephalitis, and West Nile viruses. Like other flaviviruses, Zika virus is enveloped and icosahedral and has a nonsegmented, single-stranded, 10 kb positive-sense RNA genome. It is most closely related to the Spondweni virus and is one of the two known viruses in the Spondweni virus Glade.

A positive-sense RNA genome can be directly translated into viral proteins. As in other flaviviruses, such as the similarly sized West Nile virus, the RNA genome encodes seven nonstructural proteins and three structural proteins. One of the structural proteins encapsulates the virus. The RNA genome forms a nucleocapsid along with copies of the 12-kDa capsid protein. The nucleocapsid, in turn, is enveloped within a host-derived membrane modified with two viral glycoproteins. Viral genome replication depends on the synthesis of double sided RNA from the single stranded positive sense RNA (ssRNA(+)) genome followed by transcription and replication to provide viral mRNAs and new ssRNA(+) genomes.

There are two Zika lineages: the African lineage and the Asian lineage. Phylogenetic studies indicate that the virus spreading in the Americas is 89% identical to African genotypes but is most closely related to the Asian strain that circulated in French Polynesia during the 2013-2014 outbreak.

The vertebrate hosts of the virus were primarily monkeys in a so-called enzootic mosquito-monkey-mosquito cycle, with only occasional transmission to humans. Before the current pandemic began in 2007, Zika “rarely caused recognized ‘spillover’ infections in humans, even in highly enzootic areas.” Infrequently, however, other arboviruses have become established as a human disease and spread in a mosquito-human-mosquito cycle, like the yellow fever virus and the dengue fever virus (both flaviviruses), and the chikungunya virus (a togavirus). Though the reason for the pandemic is unknown, dengue, a related arbovirus that infects the same species of mosquito vectors, is known in particular to be intensified by urbanization and globalization. Zika is primarily spread by Aedes aegypti mosquitoes and can also be transmitted through sexual contact or blood transfusions. The basic reproduction number (R₀, a measure of transmissibility) of Zika virus has been estimated to be between 1.4 and 6.6.

In 2015, news reports drew attention to the rapid spread of Zika in Latin America and the Caribbean. At that time, the Pan American Health Organization published a list of countries and territories that experienced “local Zika virus transmission” comprising Barbados, Bolivia, Brazil, Colombia, the Dominican Republic, Ecuador, El Salvador, French Guiana, Guadeloupe, Guatemala, Guyana, Haiti, Honduras, Martinique, Mexico, Panama, Paraguay, Puerto Rico, Saint Martin, Suriname, and Venezuela. By August 2016, more than 50 countries had experienced active (local) transmission of Zika virus.

Zika is primarily spread by the female Aedes aegypti mosquito, which is active mostly in the daytime, although researchers have found the virus in common Culex house mosquitoes as well. The mosquitos must feed on blood in order to lay eggs. The virus has also been isolated from a number of arboreal mosquito species in the Aedes genus, such as A. africanus, A. apicoargenteus, A. furcifer, A. hensilli, A. luteocephalus and A. vittatus, with an extrinsic incubation period in mosquitoes of about 10 days.

The true extent of the vectors is still unknown. Zika has been detected in many more species of Aedes, along with Anopheles coustani, Mansonia uniformis, and Culex perfuscus, although this alone does not incriminate them as a vector.

Transmission by A. albopictus, the tiger mosquito, was reported from a 2007 urban outbreak in Gabon where it had newly invaded the country and become the primary vector for the concomitant chikungunya and dengue virus outbreaks. There is concern for autochthonous infections in urban areas of European countries infested by A. albopictus because the first two cases of laboratory-confirmed Zika infections imported into Italy were reported from viremic travelers returning from French Polynesia.

The potential societal risk of Zika can be delimited by the distribution of the mosquito species that transmit it. The global distribution of the most cited carrier of Zika, A. aegypti, is expanding due to global trade and travel. A. aegypti distribution is now the most extensive ever recorded—across all continents including North America and even the European periphery (Madeira, the Netherlands, and the northeastern Black Sea coast). A mosquito population capable of carrying Zika has been found in a Capitol Hill neighborhood of Washington, D.C., and genetic evidence suggests they survived at least four consecutive winters in the region. The study authors conclude that mosquitos are adapting for persistence in a northern climate. The Zika virus appears to be contagious via mosquitoes for around a week after infection. The virus is thought to be infectious for a longer period of time after infection (at least 2 weeks) when transmitted via semen.

Research into its ecological niche suggests that Zika may be influenced to a greater degree by changes in precipitation and temperature than Dengue, making it more likely to be confined to tropical areas. However, rising global temperatures would allow for the disease vector to expand their range further north, allowing Zika to follow.

Zika can be transmitted from men and women to their sexual partners. As of April 2016, sexual transmission of Zika has been documented in six countries—Argentina, Chile, France, Italy, New Zealand and the United States—during the 2015 outbreak.

In 2014, Zika capable of growth in lab culture was found in the semen of a man at least two weeks (and possibly up to 10 weeks) after he fell ill with Zika fever. In 2011 a study found that a U.S. biologist who had been bitten many times while studying mosquitoes in Senegal developed symptoms six days after returning home in August 2008, but not before having unprotected intercourse with his wife, who had not been outside the U.S. since 2008. Both husband and wife were confirmed to have Zika antibodies, raising awareness of the possibility of sexual transmission. In early February 2016, the Dallas County Health and Human Services department reported that a man from Texas who had not travelled abroad had been infected after his male monogamous sexual partner had anal penetrative sex with him one day before and one day after onset of symptoms. As of February 2016, fourteen additional cases of possible sexual transmission have been under investigation, but it remained unknown whether women can transmit Zika to their sexual partners. At that time, the understanding of the “incidence and duration of shedding in the male genitourinary tract [was] limited to one case report.” Therefore, the CDC interim guideline recommended against testing men for purposes of assessing the risk of sexual transmission.

In March 2016, the CDC updated its recommendations about length of precautions for couples and advised that heterosexual couples with men who have confirmed Zika fever or symptoms of Zika should consider using condoms or not having penetrative sex (i.e., vaginal intercourse, anal intercourse, or fellatio) for at least 6 months after symptoms begin. This includes men who live in—and men who traveled to—areas with Zika. Couples with men who traveled to an area with Zika, but did not develop symptoms of Zika, should consider using condoms or not having sex for at least 8 weeks after their return in order to minimize risk. Couples with men who live in an area with Zika, but have not developed symptoms, might consider using condoms or not having sex while there is active Zika transmission in the area. The Zika virus can spread from an infected mother to her fetus during pregnancy or at delivery.

As of April 2016, two cases of Zika transmission through blood transfusions have been reported globally, both from Brazil, after which the US Food and Drug Administration (FDA) recommended screening blood donors and deferring high-risk donors for 4 weeks. A potential risk had been suspected based on a blood-donor screening study during the French Polynesian Zika outbreak, in which 2.8% (42) of donors from November 2013 and February 2014 tested positive for Zika RNA and were all asymptomatic at the time of blood donation. Eleven of the positive donors reported symptoms of Zika fever after their donation, but only three of 34 samples grew in culture.

Zika virus replicates in the mosquito's midgut epithelial cells and then its salivary gland cells. After 5-10 days, the virus can be found in the mosquito's saliva. If the mosquito's saliva is inoculated into human skin, the virus can infect epidermal keratinocytes, skin fibroblasts in the skin and the Langerhans cells. The pathogenesis of the virus is hypothesized to continue with a spread to lymph nodes and the bloodstream. Flaviviruses generally replicate in the cytoplasm, but Zika antigens have been found in infected cell nuclei.

Zika fever (also known as Zika virus disease) is an illness caused by the Zika virus. Most cases have no symptoms, but when present they are usually mild and can resemble dengue fever. Symptoms may include fever, red eyes, joint pain, headache, and a maculopapular rash. Symptoms generally last less than seven days. It has not caused any reported deaths during the initial infection. Infection during pregnancy causes microcephaly and other brain malformations in some babies. Infection in adults has been linked to Guillain-Barré syndrome (GBS). Diagnosis is by testing the blood, urine, or saliva for the presence of Zika virus RNA when the person is sick.

Prevention involves decreasing mosquito bites in areas where the disease occurs, and proper use of condoms. Efforts to prevent bites include the use of insect repellent, covering much of the body with clothing, mosquito nets, and getting rid of standing water where mosquitoes reproduce. There is no effective vaccine. Health officials recommended that women in areas affected by the 2015-16 Zika outbreak consider putting off pregnancy and that pregnant women not travel to these areas. While there is no specific treatment, paracetamol (acetaminophen) and rest may help with the symptoms. Admission to hospital is rarely necessary.

Effective vaccines have existed for several viruses of the flaviviridae family, namely yellow fever vaccine, Japanese encephalitis vaccine, and tick-borne encephalitis vaccine, since the 1930s, and dengue fever vaccine since the mid-2010s. World Health Organization (WHO) experts have suggested that the priority should be to develop inactivated vaccines and other non-live vaccines, which are safe to use in pregnant women and those of childbearing age.

As of March 2016, eighteen companies and institutions internationally were developing vaccines against Zika but a vaccine was unlikely to be widely available for about ten years. In June 2016 the FDA granted the first approval for a human clinical trial for a Zika vaccine.

The virus was first isolated in April 1947 from a rhesus macaque monkey that had been placed in a cage in the Zika Forest of Uganda, near Lake Victoria, by the scientists of the Yellow Fever Research Institute. A second isolation from the mosquito A. africanus followed at the same site in January 1948. When the monkey developed a fever, researchers isolated from its serum a “filterable transmissible agent” that was named Zika in 1948.

Zika had been known to infect humans from the results of serological surveys in Uganda and Nigeria, published in 1952: Among 84 people of all ages, 50 individuals had antibodies to Zika, and all above 40 years of age were immune. A 1952 research study conducted in India had shown a “significant number” of Indians tested for Zika had exhibited an immune response to the virus, suggesting it had long been widespread within human populations.

It was not until 1954 that the isolation of Zika from a human was published. This came as part of a 1952 outbreak investigation of jaundice suspected to be yellow fever. It was found in the blood of a 10-year-old Nigerian female with low-grade fever, headache, and evidence of malaria, but no jaundice, who recovered within three days. Blood was injected into the brain of laboratory mice, followed by up to 15 mice passages. The virus from mouse brains was then tested in neutralization tests using rhesus monkey sera specifically immune to Zika. In contrast, no virus was isolated from the blood of two infected adults with fever, jaundice, cough, diffuse joint pains in one and fever, headache, pain behind the eyes and in the joints. Infection was proven by a rise in Zika-specific serum antibodies.

From 1951 through 1983, evidence of human infection with Zika was reported from other African countries, such as the Central African Republic, Egypt, Gabon, Sierra Leone, Tanzania, and Uganda, as well as in parts of Asia including India, Indonesia, Malaysia, the Philippines, Thailand, Vietnam and Pakistan. From its discovery until 2007, there were only 14 confirmed human cases of Zika infection from Africa and Southeast Asia.

In April 2007, the first outbreak outside of Africa and Asia occurred on the island of Yap in the Federated States of Micronesia, characterized by rash, conjunctivitis, and arthralgia, which was initially thought to be dengue, chikungunya, or Ross River disease. Serum samples from patients in the acute phase of illness contained RNA of Zika. There were 49 confirmed cases, 59 unconfirmed cases, no hospitalizations, and no deaths. Between 2013 and 2014, further epidemics occurred in French Polynesia, Easter Island, the Cook Islands, and New Caledonia. On 22 March 2016 Reuters reported that Zika was isolated from a 2014 blood sample of an elderly man in Chittagong in Bangladesh as part of a retrospective study.

As of early 2016, a widespread outbreak of Zika was ongoing, primarily in the Americas. The outbreak began in April 2015 in Brazil, and has spread to other countries in South America, Central America, North America, and the Caribbean. The Zika virus reached Singapore and Malaysia in Aug 2016. In January 2016, the WHO said the virus was likely to spread throughout most of the Americas by the end of the year; and in February 2016, the WHO declared the cluster of microcephaly and Guillain-Barré syndrome cases reported in Brazil—strongly suspected to be associated with the Zika outbreak—a Public Health Emergency of International Concern. It is estimated that 1.5 million people have been infected by Zika in Brazil, with over 3,500 cases of microcephaly reported between October 2015 and January 2016.

A number of countries have issued travel warnings, and the outbreak is expected to significantly impact the tourism industry. Several countries have taken the unusual step of advising their citizens to delay pregnancy until more is known about the virus and its impact on fetal development. With the 2016 Summer Olympic Games hosted in Rio de Janeiro, health officials worldwide have voiced concerns over a potential crisis, both in Brazil and when international athletes and tourists, who may be unknowingly infected, return home and possibly spread the virus. Some researchers speculate that only one or two tourists may be infected during the three-week period, or approximately 3.2 infections per 100,000 tourists.

### II. SERMs and Non-SERM Compounds

### A. Tamoxifen

Tamoxifen, sold under the brand name Nolvadex among others, is a medication that is used to prevent breast cancer in women and treat breast cancer in women and men. It is also being studied for other types of cancer. It has been used for Albright syndrome. Tamoxifen is typically taken daily by mouth for five years for breast cancer.

Serious side effects include a small increased risk of uterine cancer, stroke, vision problems, and pulmonary embolism. Common side effects include irregular periods, weight loss, and hot flashes. It may cause harm to the baby if taken during pregnancy or breastfeeding. It is a selective estrogen-receptor modulator (SERM) and works by decreasing the growth of breast cancer cells. It is of the triphenylethylene group.

Tamoxifen was initially made in 1962 by chemist Dora Richardson. It is on the World Health Organization's List of Essential Medicines, the most effective and safe medicines needed in a health system. Tamoxifen is available as a generic medication. The wholesale price in the developing world is about 0.07 to 0.23 USD per day. In the United States it costs about 1 USD a day.

Tamoxifen is currently used for the treatment of both early and advanced estrogen receptor-positive (ER-positive or ER+) breast cancer in pre- and post-menopausal women. Additionally, it is the most common hormone treatment for male breast cancer. It is also approved by the FDA for the prevention of breast cancer in women at high risk of developing the disease. It has been further approved for the reduction of contralateral (in the opposite breast) cancer. The use of tamoxifen is recommended for 10 years.

In 2006, the large STAR clinical study concluded that raloxifene is equally effective in reducing the incidence of breast cancer, but after an average 4-year follow-up, although the difference was not statistically significant, there were 36% fewer uterine cancers and 29% fewer blood clots in women taking raloxifene than in women taking tamoxifen.

Tamoxifen is used to treat infertility in women with anovulatory disorders. It is given at days 3 to 7 of a woman's cycle. Tamoxifen improves fertility in males with infertility by disinhibiting the hypothalamic-pituitary-gonadal axis (HPG axis) via ER antagonism and thereby increasing the secretion of luteinizing hormone (LH) and follicle-stimulating hormone (FSH) and increasing testicular testosterone production.

Tamoxifen is used to prevent or treat gynecomastia. It is taken as a preventative measure in small doses or used at the onset of any symptoms such as nipple soreness or sensitivity. Other drugs are taken for similar purposes such as clomifene and the anti-aromatase drugs which are used in order to try to avoid the hormone-related adverse effects.

Occasionally tamoxifen is used in treatment of the rare conditions of retroperitoneal fibrosis and idiopathic sclerosing mesenteritis.

A report in September 2009 from Health and Human Services' Agency for Healthcare Research and Quality suggests that tamoxifen, raloxifene, and tibolone used to treat breast cancer significantly reduce invasive breast cancer in midlife and older women, but also increase the risk of adverse side effects. Some cases of lower-limb lymphedema have been associated with the use of tamoxifen, due to the blood clots and deep vein thrombosis (DVT) that can be caused by this medication. Resolution of the blood clots or DVT is needed before lymphedema treatment can be initiated. One in vitro study in 2007 and later an in vivo study in 2008 have shown that tamoxifen induces apoptosis in growth plate chondrocytes, reduces serum insulin-like growth factor 1 (IGF-1) levels and causes persistent retardation of longitudinal and cortical radial bone growth in young male rats, leading the researchers to express concern giving tamoxifen to growing individuals.

A beneficial side effect of tamoxifen is that it prevents bone loss by acting as an ER agonist (i.e., mimicking the effects of estrogen) in this cell type. Therefore, by inhibiting osteoclasts, it prevents osteoporosis. When tamoxifen was launched as a drug, it was thought that tamoxifen would act as an ER antagonist in all tissue, including bone, and therefore it was feared that it would contribute to osteoporosis. It was therefore very surprising that the opposite effect was observed clinically. Hence tamoxifen's tissue selective action directly led to the formulation of the concept of SERMs. In contrast tamoxifen appears to be associated with bone loss in premenopausal women who continue to menstruate after adjuvant chemotherapy.

Tamoxifen is a SERM. Even though it is an antagonist in breast tissue it acts as partial agonist on the endometrium and has been linked to endometrial cancer in some women. Therefore, endometrial changes, including cancer, are among tamoxifen's side effects. With time, risk of endometrial cancer may be doubled to quadrupled, which is a reason tamoxifen is typically only used for 5 years.

The American Cancer Society lists tamoxifen as a known carcinogen, stating that it increases the risk of some types of uterine cancer while lowering the risk of breast cancer recurrence. The ACS states that its use should not be avoided in cases where the risk of breast cancer recurrence without the drug is higher than the risk of developing uterine cancer with the drug.

Tamoxifen treatment of postmenopausal women is associated with beneficial effects on serum lipid profiles. However, long-term data from clinical trials have failed to demonstrate a cardioprotective effect. For some women, tamoxifen can cause a rapid increase in triglyceride concentration in the blood. In addition, there is an increased risk of thromboembolism especially during and immediately after major surgery or periods of immobility. Use of tamoxifen has been shown to slightly increase the risk of deep vein thrombosis, pulmonary embolism, and stroke. Tamoxifen is also a cause of fatty liver, otherwise known as steatorrhoeic hepatosis or steatosis hepatis.

Tamoxifen-treated breast cancer patients show evidence of reduced cognition, a major side effect of tamoxifen, and semantic memory scores. However, memory impairment in patients treated with tamoxifen was less severe compared with those treated with anastrozole (an aromatase inhibitor). A significant number of tamoxifen-treated breast cancer patients experience a reduction of libido.

While tamoxifen has been shown to antagonize the actions of estrogen in tissues such as the breast, its effects in other tissues such as bones has not been documented fully. There have been studies done in mice showing tamoxifen mimic the effects of estrogen on bone metabolism and skeletal growth, thus increasing the possibility of premature bone fusion. This effect would be less of a concern in adults who have stopped growing.

Patients with variant forms of the gene CYP2D6 (also called simply 2D6) may not receive full benefit from tamoxifen because of too slow metabolism of the tamoxifen prodrug into its active metabolites. On 18 Oct. 2006, the Subcommittee for Clinical Pharmacology recommended relabeling tamoxifen to include information about this gene in the package insert. Certain CYP2D6 variations in breast cancer patients lead to a worse clinical outcome for tamoxifen treatment. Genotyping therefore has the potential for identification of women who have these CYP2D6 phenotypes and for whom the use of tamoxifen is associated with poor outcomes.

Recent studies suggest that taking the selective serotonin reuptake inhibitors (SSRIs) antidepressants paroxetine (Paxil), fluoxetine (Prozac), and sertraline (Zoloft) can decrease the effectiveness of tamoxifen, as these drugs compete for the CYP2D6 enzyme which is needed to metabolize tamoxifen into its active forms. A U.S. study presented at the American Society of Clinical Oncology's annual meeting in 2009 found that after two years, 7.5% of women who took only tamoxifen had a recurrence, compared with 16% who took either paroxetine, fluoxetine or sertraline, drugs considered to be the most potent CYP2D6 inhibitors. That difference translates to a 120% increase in the risk of breast cancer recurrence. Patients taking the SSRIs; Celexa (citalopram), Lexapro (escitalopram), and Luvox (fluvoxamine), did not have an increased risk of recurrence, due to their lack of competitive metabolism for the CYP2D6 enzyme. A newer study demonstrated a clearer and stronger effect from paroxetine in causing the worst outcomes. Patients treated with both paroxetine and tamoxifen have a 67% increased risk of death from breast cancer, from 24% to 91%, depending on the duration of coadministration.

Recent research has shown that 7-10% of women with breast cancer may not receive the full medical benefit from taking tamoxifen due to their genetic make-up. DNA Drug Safety Testing can examine DNA variations in the CYP2D6 and other important drug processing pathways. More than 20% of all clinically used medications are metabolized by CYP2D6 and knowing the CYP2D6 status of a person can help the doctor with the future selection of medications. Other molecular biomarkers may also be used to select appropriate patients likely to benefit from tamoxifen.

Tamoxifen itself is a prodrug, having relatively little affinity for its target protein, the estrogen receptor (ER). It is metabolized in the liver by the cytochrome P450 isoform CYP2D6 and CYP3A4 into active metabolites such as afimoxifene (4-hydroxytamoxifen; TAM-OH) and endoxifen (N-desmethyl-4-hydroxytamoxifen) which have 30 to 100 times greater affinity for the ER than tamoxifen itself. These active metabolites compete with estrogen in the body for binding to the ER. In breast tissue, TAM-OH acts as an ER antagonist so that transcription of estrogen-responsive genes is inhibited. Tamoxifen has 7% and 6% of the affinity of estradiol for the ERα and ERβ, respectively, whereas TAM-OH has 178% and 338% of the affinity of estradiol for the ERα and ERβ.

TAM-OH binds to ER, the ER/tamoxifen complex recruits other proteins known as co-repressors and then binds to DNA to modulate gene expression. Some of these proteins include NCoR and SMRT. Tamoxifen function can be regulated by a number of different variables including growth factors. Tamoxifen needs to block growth factor proteins such as ErbB2/HER2 because high levels of ErbB2 have been shown to occur in tamoxifen resistant cancers. Tamoxifen seems to require a protein PAX2 for its full anticancer effect. In the presence of high PAX2 expression, the tamoxifen/ER complex is able to suppress the expression of the pro-proliferative ERBB2 protein. In contrast, when AIB-1 expression is higher than PAX2, tamoxifen/ER complex upregulates the expression of ERBB2 resulting in stimulation of breast cancer growth.

TAM-OH binds to ER competitively (with respect to the endogenous agonist estrogen) in tumor cells and other tissue targets, producing a nuclear complex that decreases DNA synthesis and inhibits estrogen effects. It is a nonsteroidal agent with potent antiestrogenic properties which compete with estrogen for binding sites in breast and other tissues. Tamoxifen causes cells to remain in the Go and Gi phases of the cell cycle. Because it prevents (pre)cancerous cells from dividing but does not cause cell death, tamoxifen is cytostatic rather than cytocidal.

Norendoxifen (N,N-didesmethyl-4-hydroxytamoxifen), another active metabolite of tamoxifen, has been found to act as a potent competitive aromatase inhibitor (IC₅₀=90 nM), and may also be involved in its antiestrogenic activity.

Tamoxifen has been found to decrease insulin-like growth factor 1 levels by 17 to 38% in women and men.

Tamoxifen has also been studied in several additional indications. Tamoxifen has been proposed as part of a treatment plan for Riedel's thyroiditis. Tamoxifen has been shown to be effective in the treatment of mania in patients with bipolar disorder by blocking protein kinase C (PKC), an enzyme that regulates neuron activity in the brain. Researchers believe PKC is over-active during the mania in bipolar patients. In McCune-Albright syndrome (MAS) tamoxifen has been used to treat premature puberty and the consequences of premature puberty. Tamoxifen has been seen to decrease rapid bone maturation which is the result of excessive estrogen and alter predicted adult height (PAH). The same effects have also been seen in short pubertal boys.

### B. Other SERMs

Selective estrogen receptor modulators (SERMs), like TAM, are a class of drugs that act on the estrogen receptor (ER). A characteristic that distinguishes these substances from pure ER agonists and antagonists (that is, full agonists and silent antagonists) is that their action is different in various tissues, thereby granting the possibility to selectively inhibit or stimulate estrogen-like action in various tissues. SERMs are used for various estrogen-related diseases. Including treatment of ovulatory dysfunction in the management of infertility, treatment and prevention of postmenopausal osteoporosis, treatment and reduction in risk of breast cancer and treatment of dyspareunia due to menopause. SERM is also used in combination with conjugated estrogens indicated for the treatment of estrogen deficiency symptoms, and vasomotor symptoms associated with menopause. SERMs are used dependent on their pattern of action in various tissues. Other SERMs include Anordin, Bazedoxifene, Broparestrol, Clomifene, Cyclofenil, Laxofoxifene, Ormeloxifene, Ospemifene, Raloxifene and Toremifene.

Methylpiperidinopyrazole (MPP) is a synthetic, nonsteroidal, and highly selective antagonist of ERa that is used in scientific research to study the function of this receptor. It has 200-fold selectivity for ERa over ERr3 and 1000-fold selectivity for blocking ERa-mediated gene transcription relative to that of ERP.

### C. Non-SERMs

CALP-1. CALP-1 is a cell-permeable calmodulin (CaM) peptide agonist that binds to the EF-liand/Ca²′-binding site; produces CaM-dependent activation of phosphodiesterase. Also binds to cytoplasmic sites on other Ca²⁺ channels, including NMDA and HIV-1 gp120-activated channels, inhibiting Ca²⁺-mediated cytotoxicity and apoptosis (IC₅₀=52 μM). Shown to protect pancreatic acinar cells from gossypol (Cat.No. 1964) induced necrosis. Inhibits VLA-5-mediated adhesion of mast cells to fibronectin in vitro and attenuates inflammatory cell influx in guinea pig lung in vivo. The sequence is Val-Ala-Ile-Thr-Val-Leu-Val-Lys.

A7. A-7 is a potent, cell-permeable, calmodulin antagonist which inhibits calmodulin-activated PDE activity with an IC₅₀ of 3 μM. This compound was first discovered in 1983 (PMID: 6139736).

PMA. Phorbol is a natural, plant-derived organic compound. It is a member of the tigliane family of diterpenes. Phorbol was first isolated in 1934 as the hydrolysis product of croton oil, which is derived from the seeds of the purging croton, Croton tiglium. Various esters of phorbol have important biological properties, the most notable of which is the capacity to act as tumor promoters through activation of protein kinase C. They mimic diacylglycerols, glycerol derivatives in which two hydroxyl groups have reacted with fatty acids to form esters. The most common and potent phorbol ester is 12-O-tetradecanoylphorbol-13-acetate (TPA), also called phorbol-12-myristate-13-acetate (PMA), which is used as a biomedical research tool in contexts such as models of carcinogenesis.

Chelerythrine. Chelerythrine is a benzophenanthridine alkaloid present in the plant Chelidonium majus (greater celandine). It is a potent, selective, and cell-permeable protein kinase C inhibitor in vitro. And an efficacious antagonist of G-protein-coupled CB1 receptors. It is also found in the plants Zanthoxylum clava-herculis and Zanthoxylum rhoifolium, exhibiting antibacterial activity against Staphylococcus aureus and other human pathogens.

PHTPP. PHTPP is a synthetic, nonsteroidal, and highly selective antagonist of ERβ that is used in scientific research to study the function of this receptor. It possesses 36-fold selectivity for ERβ over ERα and is a silent antagonist of ERβ. The structure is shown below:

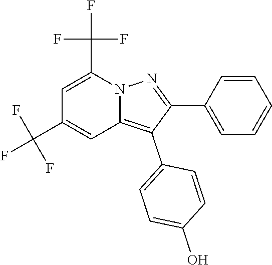 [SMILES: Oc1ccc(-c2c(-c3ccccc3)nn3c(C(F)(F)F)cc(C(F)(F)F)cc23)cc1]

Tariquidar. Tariquidar is a P-glycoprotein inhibitor undergoing research as an adjuvant against multidrug resistance in cancer. It is an anthranilamide derivative with multidrug resistance properties. Tariquidar non-competitively binds to the p-glycoprotein transporter, thereby inhibiting transmembrane transport of anticancer drugs.

S-Equol. Equol (4′,7-isoflavandiol) is an isoflavandiol estrogen metabolized from daidzein, a type of isoflavone found in soybeans and other plant sources, by bacterial flora in the intestines. While endogenous estrogenic hormones such as estradiol are steroids, equol is a nonsteroidal estrogen. However, only about 30-50% of people have intestinal bacteria that make equol. Equol can exist in two enantiomeric forms, (S)-equol and (R)-equol. (S)-Equol preferentially binds estrogen receptor beta. Under the code name AUS-131, (S)-equol is under development for the treatment of menopausal symptoms such as hot flashes and benign prostatic hyperplasia

### III. Treatment/Prevention of Zika Virus Infection

### A. Formulation and Administration

The present disclosure provides pharmaceutical compositions comprising TAM, TAM-OH, other selective estrogen receptor modulators or a compound selected from CALP-1, A7, PMA, Chelerythrine, PHTPP, Tariquidar or S-Equol. Such compositions comprise a prophylactically or therapeutically effective amount of an agent and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

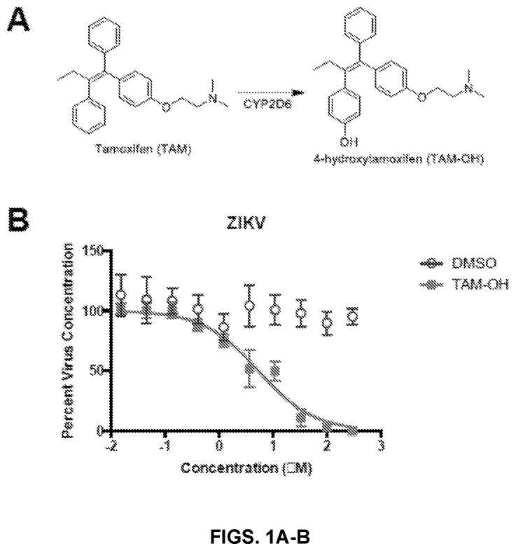

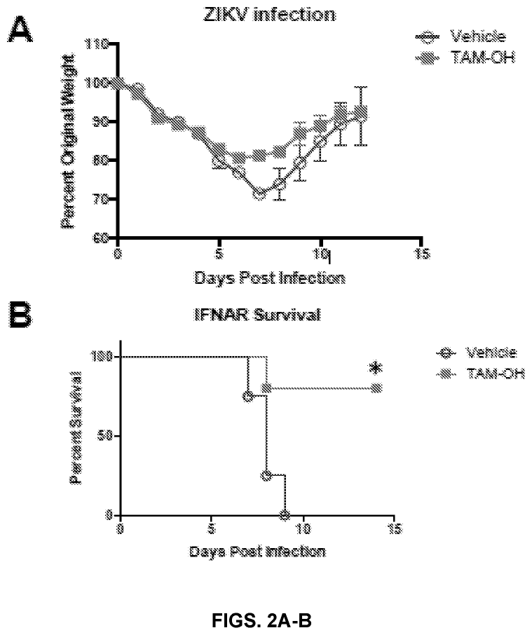

The selective estrogen receptor modulator (SERM) tamoxifen (TAM) (FIG. 1A) was shown to have antiviral activity against ZIKV grown in Vero cells (FIG. 1B). Preliminary in vivo data for the efficacy of TAM-OH shows that three doses (1 mg/animal/day) of TAM-OH delivered intraperitoneally, starting one day prior to infection decreases morbidity as demonstrated by less weight loss and increases survival (FIGS. 2A-B).

### Example 2

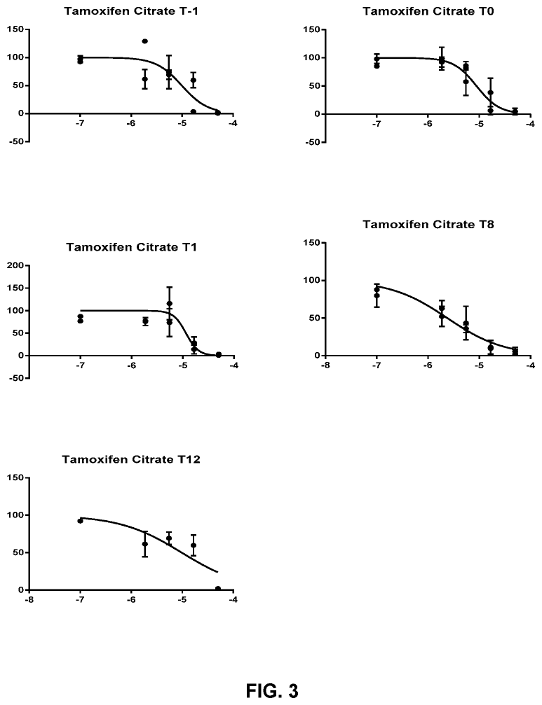

Tamoxifen citrate does not directly act on the viral particles and has a slow action time (EC₅₀ increases from T1 to T8). It does not require pre-incubation, so rather than preventing cell entry, it acts on an internal pathway of the cell which prevents viral replication (FIG. 3).

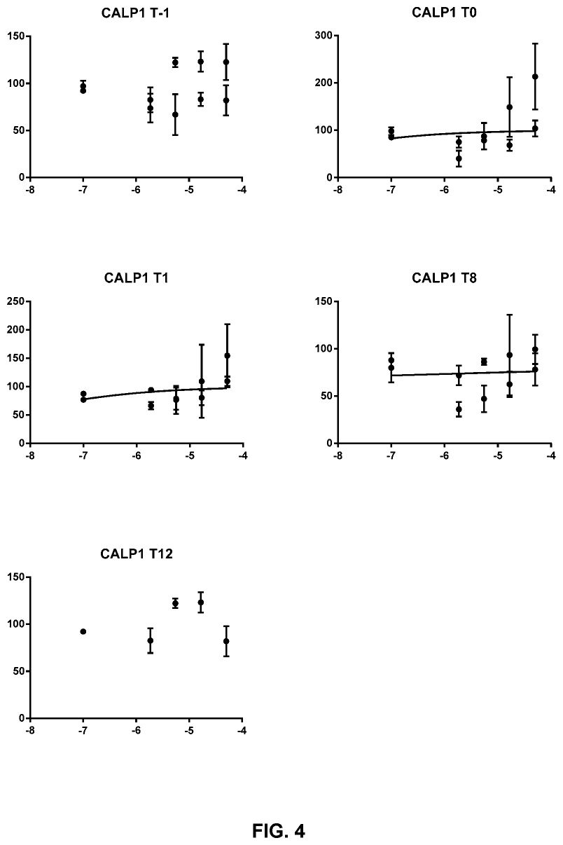

CALP1 was shown to increase viral growth during some of the assays performed, but those results were inconsistent and need further verification. It may provide some insight into a mechanism if verified, since CALP1 is a calmodulin agonist and its counterpart, A-7 Hydrochloride is a calmodulin antagonist which impedes viral replication (FIG. 4).

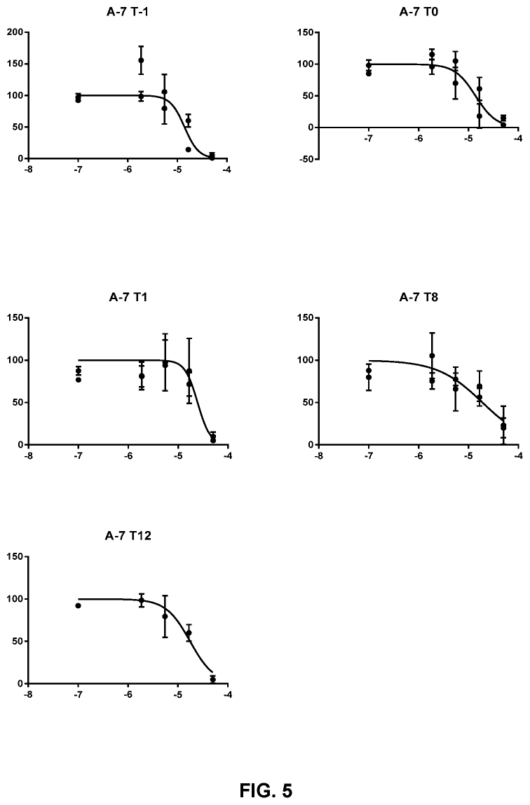

A-7 is a calmodulin antagonist which may be related to viral replication based on an increase in viral replication when virus was treated with CALP1, a calmodulin agonist. A-7 does not directly act on the viral particles and has a slow action time (EC₅₀ increases from T1 to T8). It does not require pre-incubation, so rather than preventing cell entry, it acts on an internal pathway of the cell which prevents viral replication (FIG. 5).

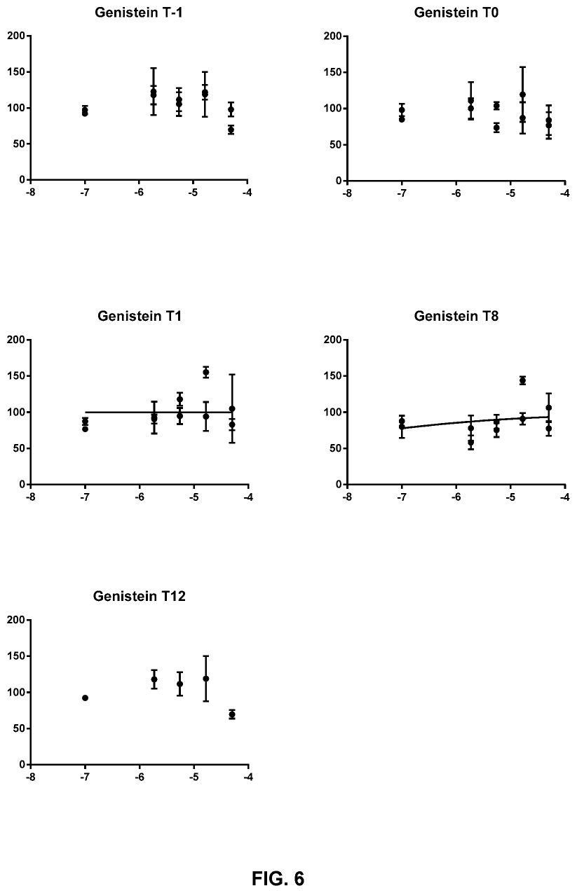

Genistein had no effect on Zika virus over the timescale of these experiments (FIG. 6).

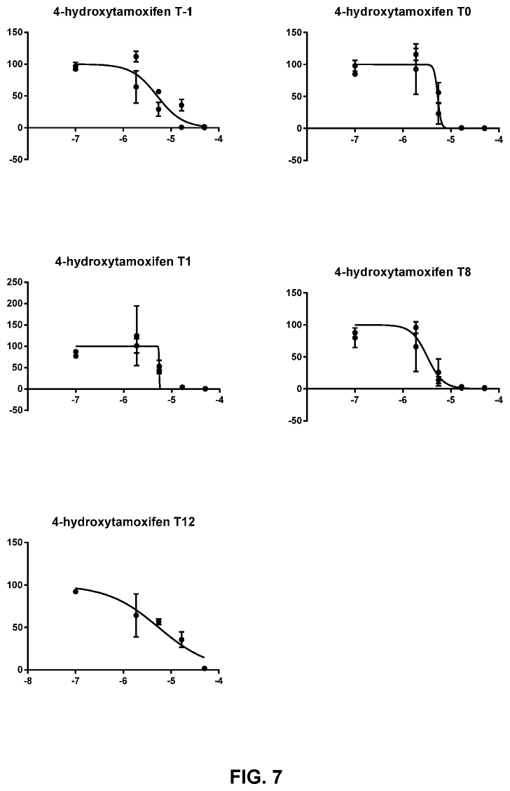

4-OH Tamoxifen does not directly act on the viral particles and has a slow action time (EC₅₀ increases from T1 to T8). It does not require pre-incubation, so rather than preventing cell entry, it acts on an internal pathway of the cell which prevents viral replication (FIG. 7).

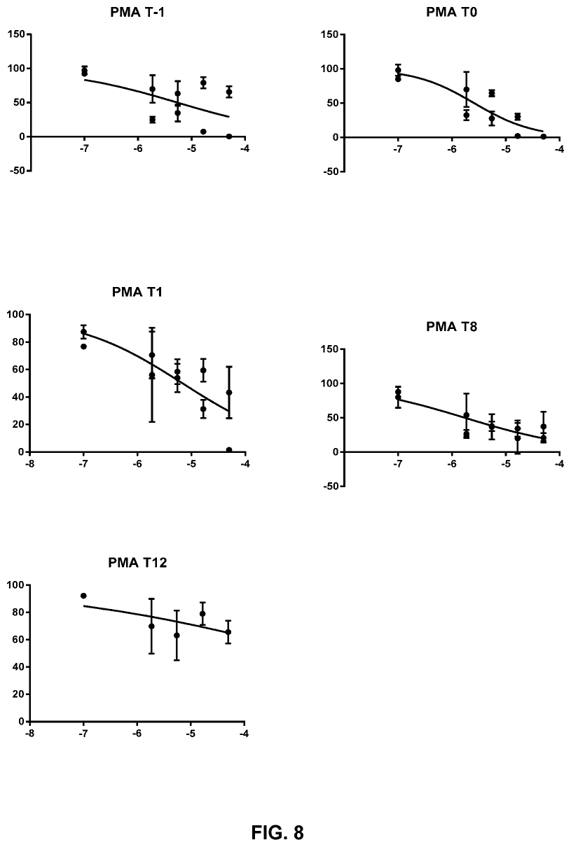

Phorbol 12-Myristate 13-Acetate (PMA) does not directly act on the viral particles and has a slow action time in Vero cells (EC₅₀ increases from T8 to T12). It does not require pre-incubation, so rather than preventing cell entry, it acts on an internal pathway of the cell which diminishes viral replication (although it does not completely kill virus through this mechanism even at high concentrations) (FIG. 8).

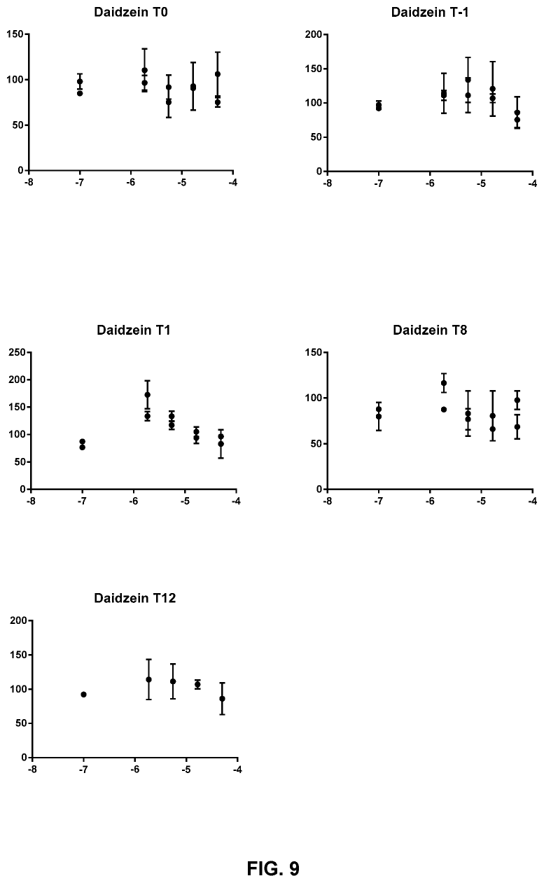

Daidzein had no effect on Zika virus over the timescale of these experiments (FIG. 9).

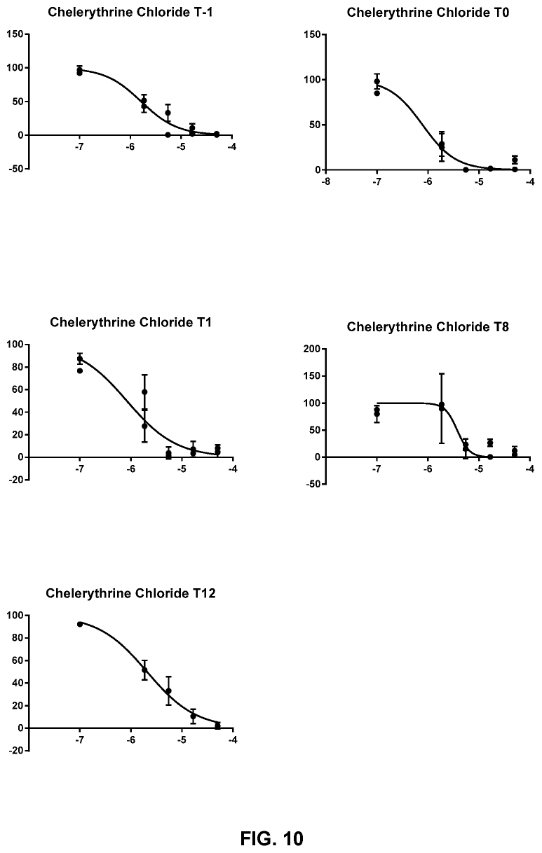

Chelerythrine Chloride does not directly act on the viral particles and has a fast action time (EC₅₀ does not change substantially from T-1 to T12). It does not require pre-incubation, so rather than preventing cell entry, it acts on an internal pathway of the cell which prevents viral replication (FIG. 10).

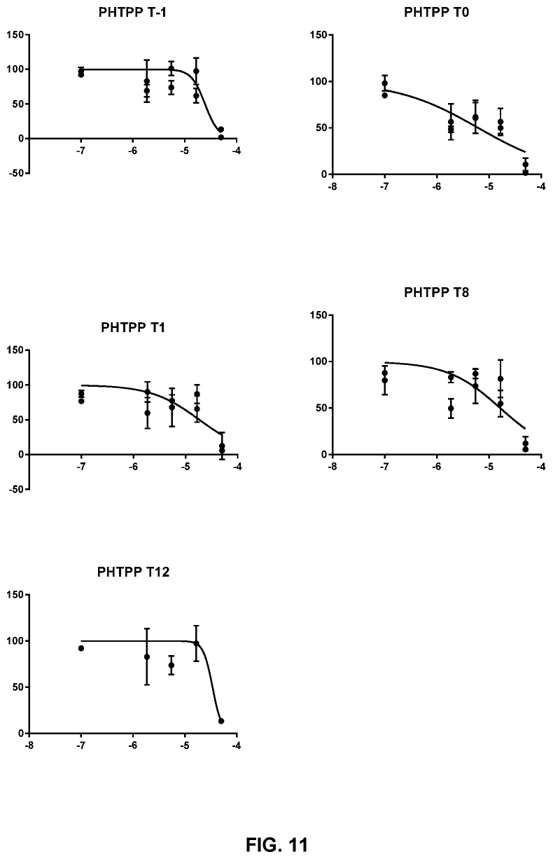

PHTPP does not directly act on the viral particles and any virus reduction is simply attributed to cell death (foci reduction is only observed in the highest concentration, which is lethal to cells as shown in the crystal violet assay) (FIG. 11).

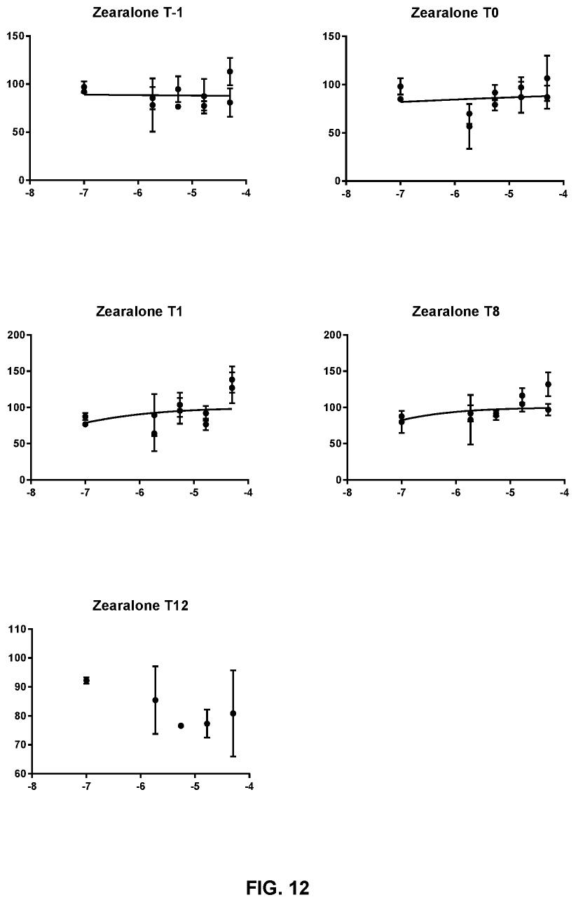

Zearalanone had no effect on Zika virus over the timescale of these experiments (FIG. 12).

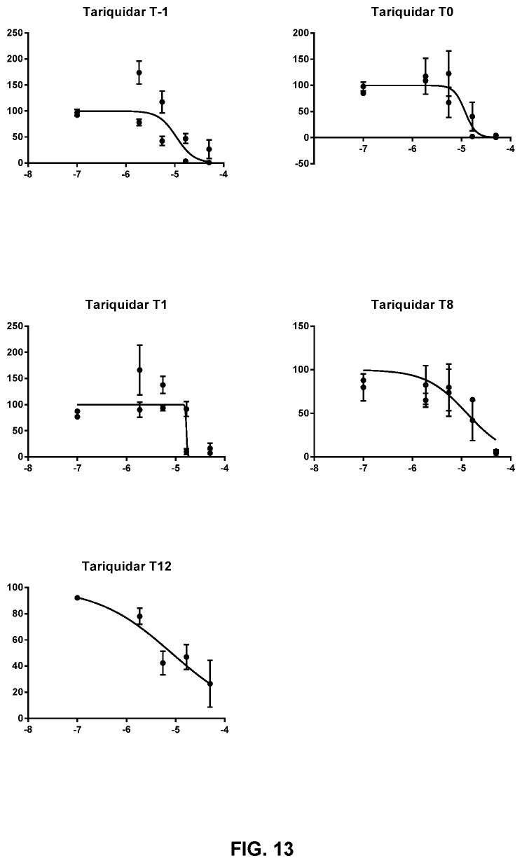

Tariquidar does not directly act on the viral particles has a fast action time (EC₅₀ does not change substantially from T-1 to T12). It does not require pre-incubation, so rather than preventing cell entry, it acts on an internal pathway of the cell which prevents viral replication (FIG. 13).

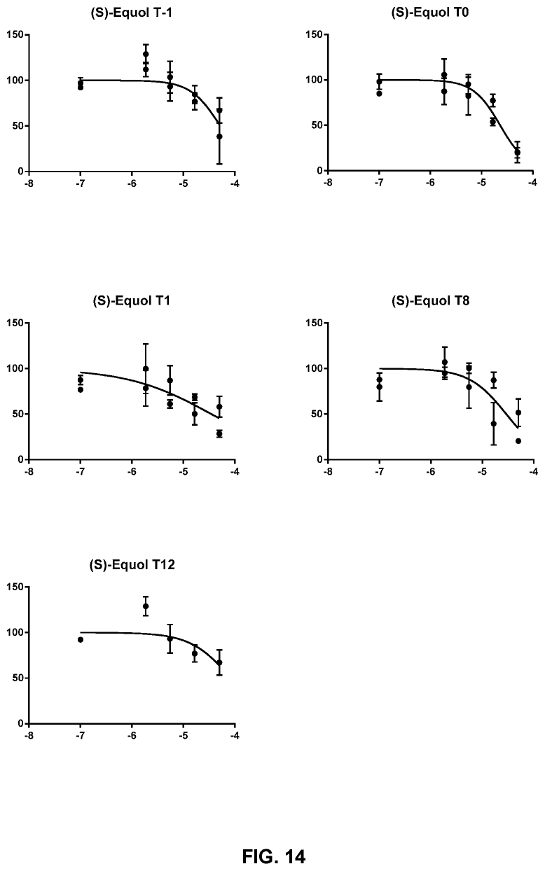

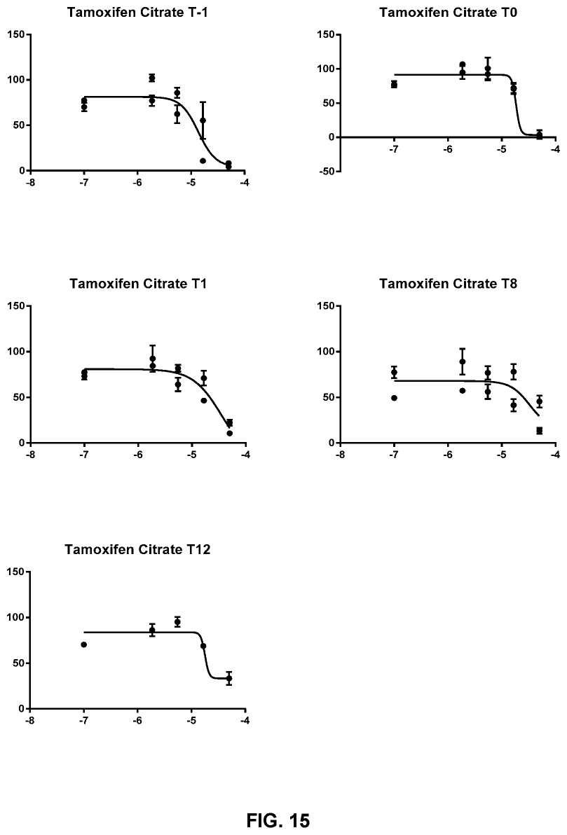

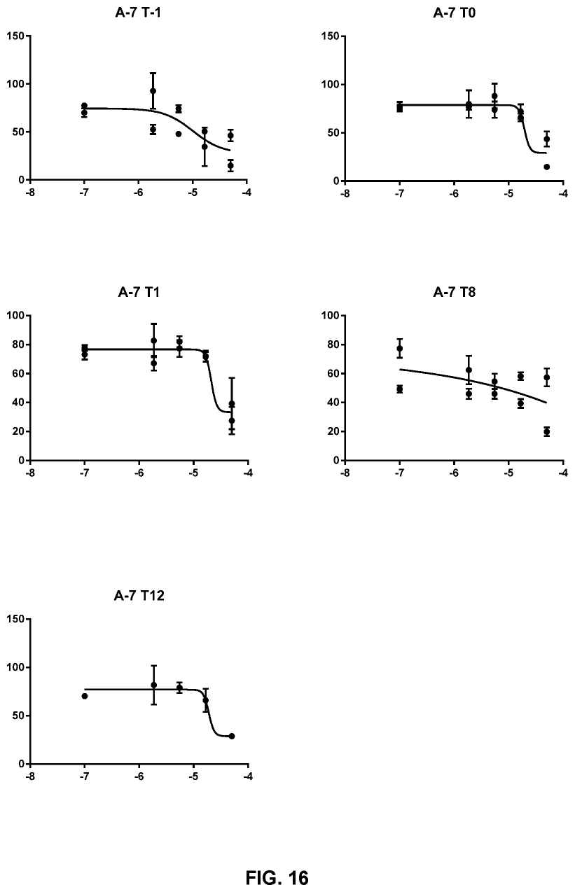

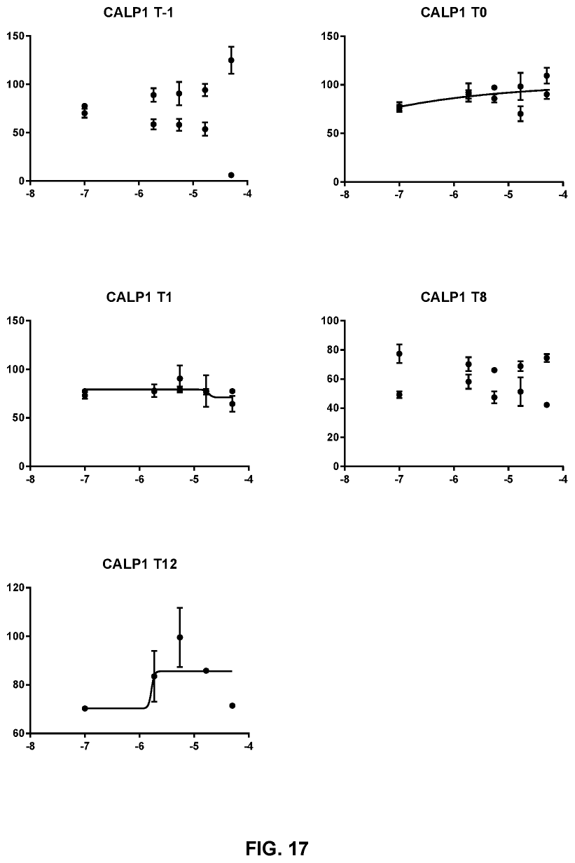

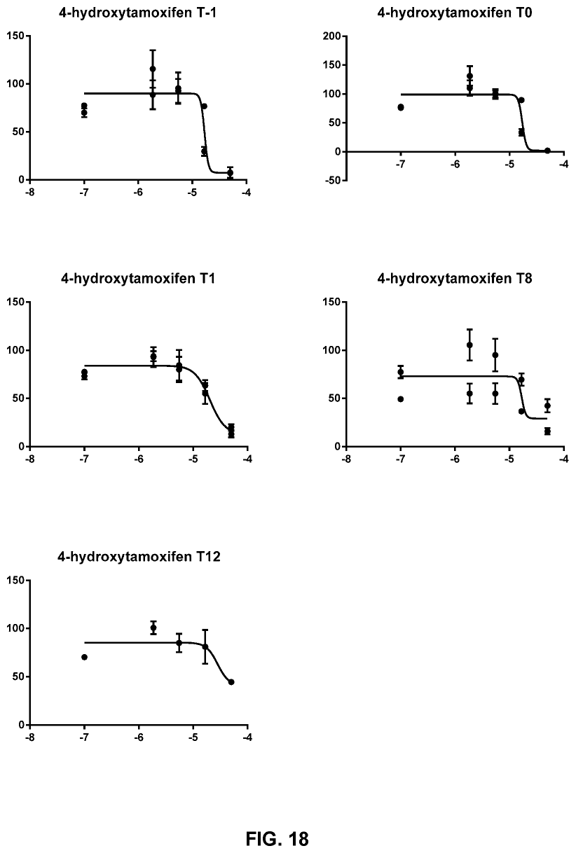

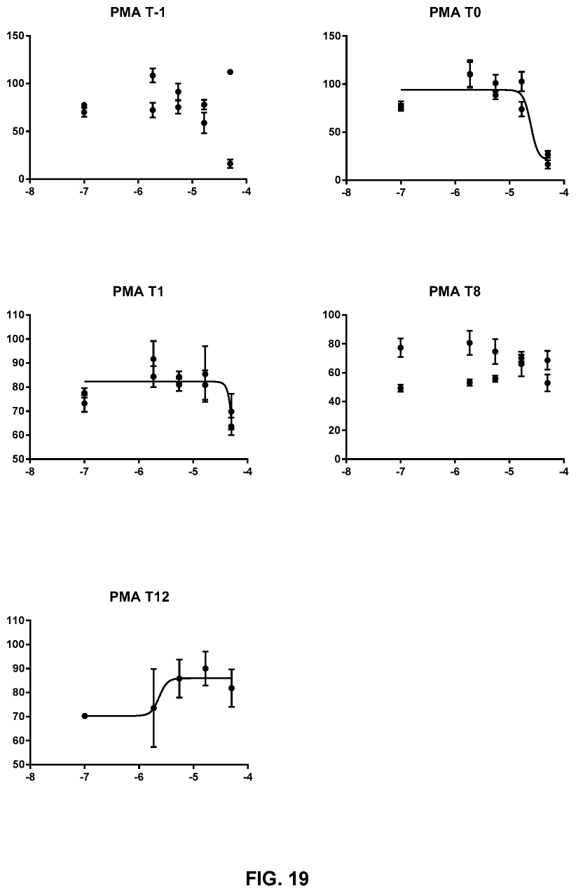

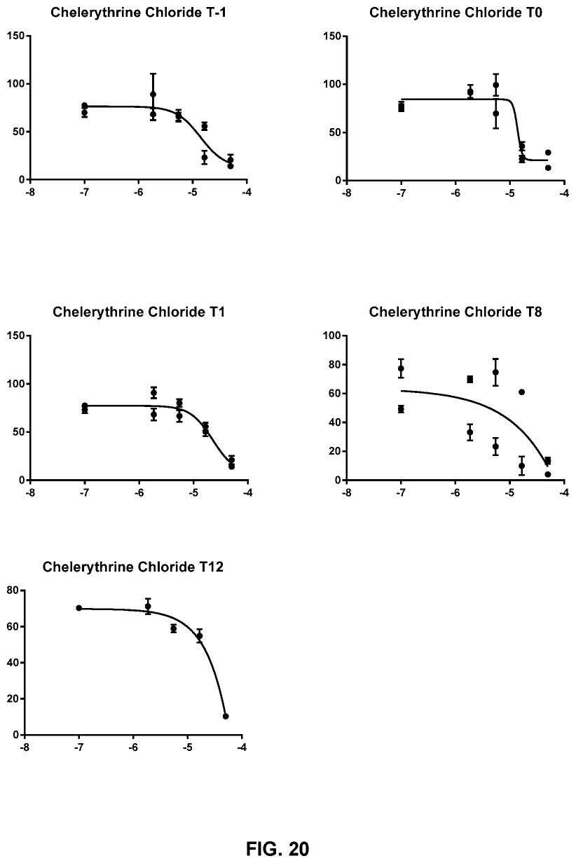

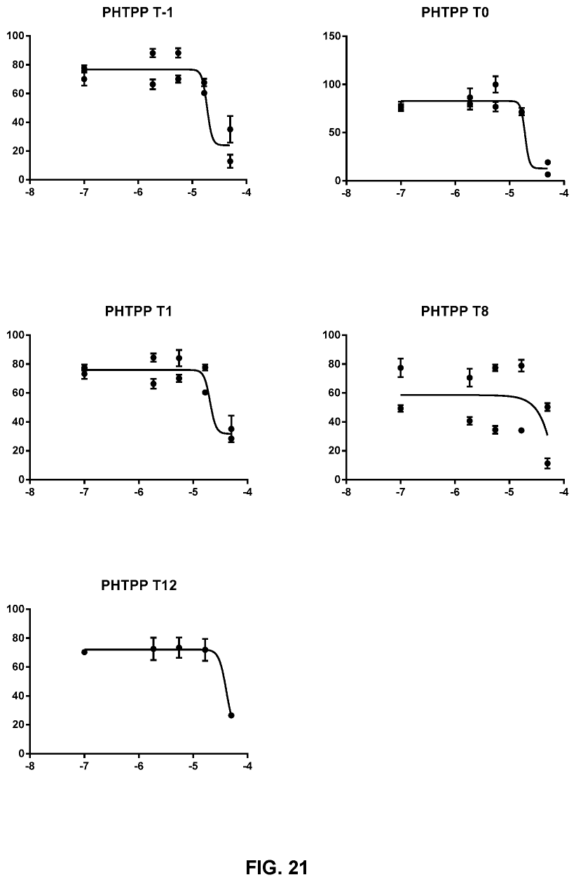

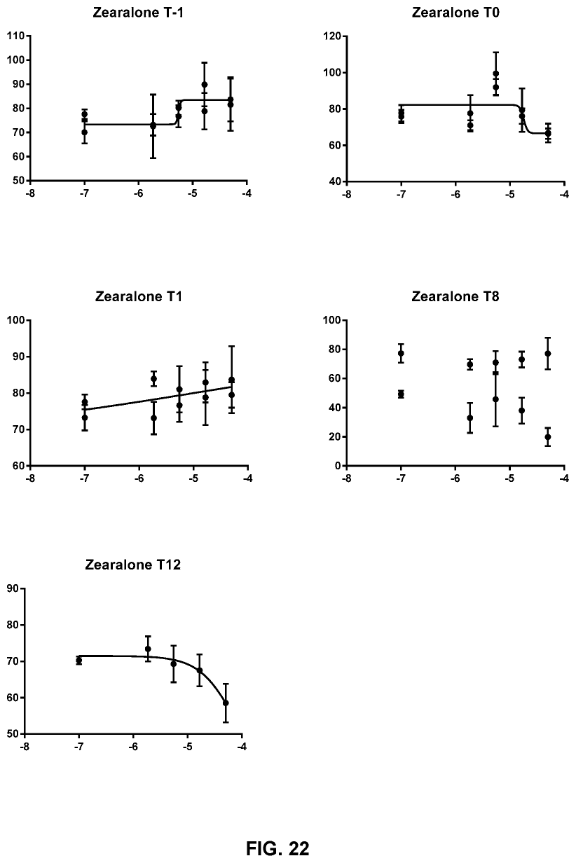

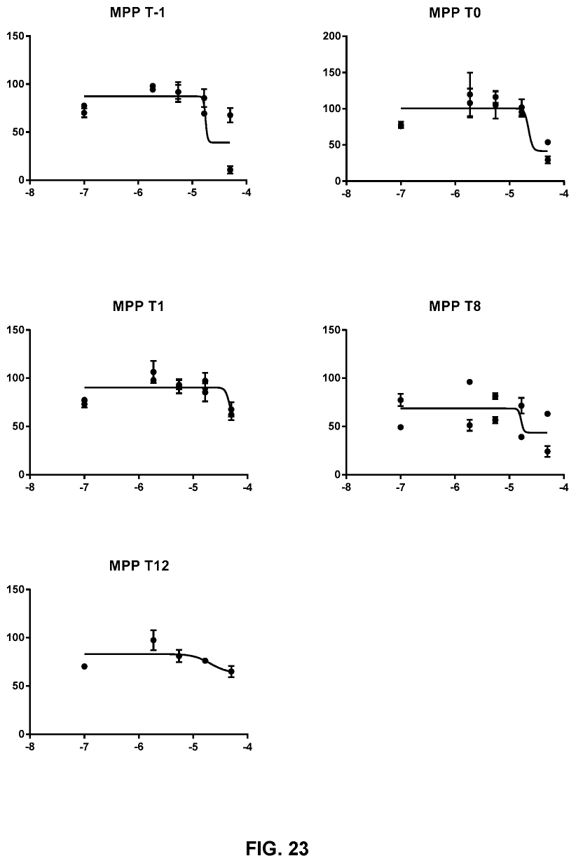

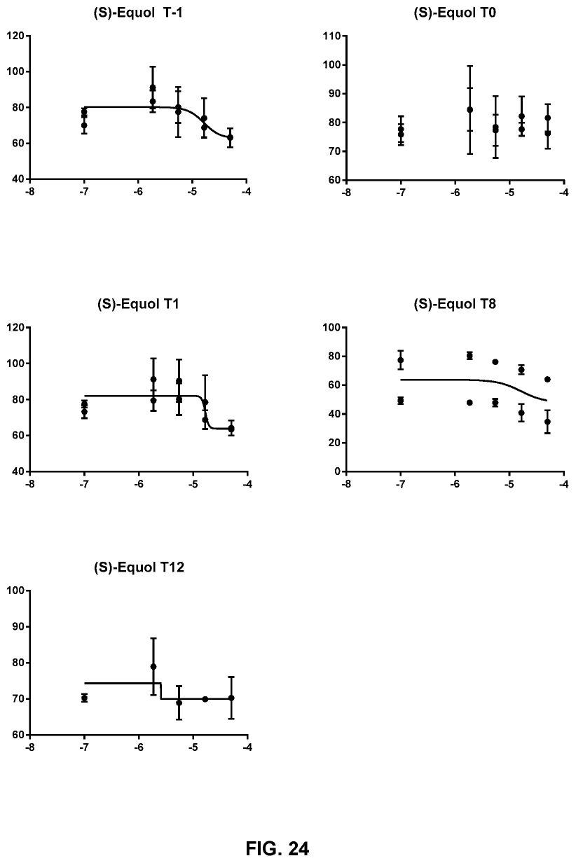

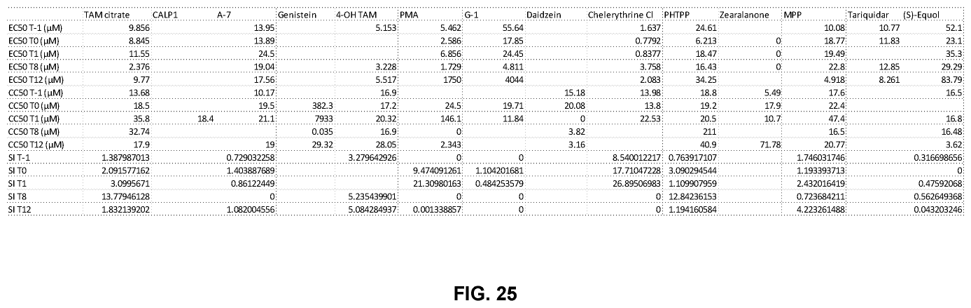

(S)-Equol does not directly act on the viral particles and has a slow action time in Vero cells (EC₅₀ increases from T8 to T12). It does not require pre-incubation, so rather than preventing cell entry, it acts on an internal pathway of the cell which diminishes viral replication (although it does not completely kill virus through this mechanism even at high concentrations) (FIG. 14).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.

Araujo et al., Brain 139, 2122-2130, 2016.

Duffy et al., N. Engl. J. Med. 360, 2536-2543, 2009.

Gatherer, D. & Kohl, A., J. Gen. Virol. 97, 269-73, 2016.

Musso et al., Clin. Microbiol. Infect. 20, 0595-0596, 2014.

Oehler et al., Euro Surveill. Bull. Eur. sur les Mal. Transm.=Eur. Commun. Dis. Bull. 19, 7-9, 2014.

Watashi et al., J. Biol. Chem. 282(45):32765-72, 2007.

1. A method of treating a subject infected with Zika virus (ZIKV) or reducing the likelihood of infection of a subject at risk of contracting ZIKV, comprising delivering to said subject a selective estrogen receptor modulator (SERM)_or a compound selected from CALP-1, A7, PMA, Chelerythrine, PHTPP, Tariquidar or S-Equol.

2. The method of claim 1, wherein the SERM is MPP, Tamoxifen or 4-hydroxy Tamoxifen.

3. The method of claim 1, wherein said subject is a pregnant female, a sexually active female, or a female undergoing fertility treatments, such as a human female.

4. The method of claim 1, wherein delivering comprises administering 4-hydroxy Tamoxifen.

5. The method of claim 1, wherein delivering comprises administering Tamoxifen, which is converted to 4-hydroxy Tamoxifen in said subject.

6. The method of claim 1, wherein delivering comprises oral, intra-venous or intra-arterial administration.

7. The method of claim 1, further comprising administering to said subject a second ZIKV therapy.

8. The method of claim 7, wherein the second ZIKV therapy is a second SERM or palliative care.

9. The method of claim 1, wherein said SERM or a compound selected from CALP-1, A7, PMA, Chelerythrine, PHTPP, Tariquidar or S-Equol is administered prior to infection or after infection.

10. The method of claim 1, wherein said SERM or a compound selected from CALP-1, A7, PMA, Chelerythrine, PHTPP, Tariquidar or S-Equol is administered after infection.

11. A method of protecting the health of a placenta and/or fetus of a pregnant female subject infected with or at risk of infection with Zika virus (ZIKV) comprising delivering to said subject a selective estrogen receptor modulator (SERM) or a compound selected from CALP-1, A7, PMA, Chelerythrine, PHTPP, Tariquidar or S-Equol.

12. The method of claim 11, wherein the SERM is MPP, Tamoxifen or 4-hydroxy Tamoxifen.

13. The method of claim 11, wherein said pregnant female subject is a human female.

14. The method of claim 11, wherein delivering comprises administering 4-hydroxy Tamoxifen.

15. The method of claim 11, wherein delivering comprises administering Tamoxifen, which is converted to 4-hydroxy Tamoxifen in said pregnant female subject.

16. The method of claim 11, wherein delivering comprises oral, intra-venous or intra-arterial administration.

17. The method of claim 11, further comprising administering to said subject a second ZIKV therapy.

18. The method of claim 17, wherein the second ZIKV therapy is a second SERM or palliative care.

19. The method of claim 11, wherein said SERM or a compound selected from CALP-1, A7, PMA, Chelerythrine, PHTPP, Tariquidar or S-Equol is administered prior to infection or after infection.

20. The method of claim 11, wherein the SERM or a compound selected from CALP-1, A7, PMA, Chelerythrine, PHTPP, Tariquidar or S-Equol increases the size of the placenta as compared to an untreated control and/or reduces viral load and/or pathology of the fetus as compared to an untreated control.
